(19) United States
(12) Patent Application Publication
(10) Pub. No.: US 2007/0184105 A1
(43) Pub. Date: Aug. 9, 2007

(54) COMSPOSITION FOR STABLIZING EPIGALLOCATECHIN GALLATE (EGCG) IN WATER PHASE AND PREPARATION METHOD THEREOF

(75) Inventors: Chul-Hwan KIM, Daejeon 302-765 (KR); Kyung-Hee KIM, Seoul 139-792 (KR); Hyun-Nam YOON, New Jersey, NJ (US)

Correspondence Address: MARGER JOHNSON & MCCOLLOM, P.C., 210 SW MORRISON STREET, SUITE 400, PORTLAND, OR, 97204, US

(73) Assignee: DPI SOLUTIONS, INC., Daejeon 305-345 (KR)

(21) Appl. No.: 10/598,865

(22) PCT Filed: Mar. 15, 2005
(86) PCT No.: PCT/KR05/00747

§ 371 Date: Sep. 13, 2006

(30) Foreign Application Priority Data
Mar. 16, 2004 (KR) 2004-0017734

(51) Int. Cl. A61K 31/353; A61K 9/20

(52) U.S. Cl. 424/464; 514/456

(57) ABSTRACT

Disclosed herein are a composition for stabilizing Epigallocatechin gallate (EGCG) in water phase comprising 0.1-25.0% by weight of Epigallocatechin gallate, 0.1-5.0% by weight of a cationic polymer, an anionic polymer or a mixture thereof, 0.1-10.0% by weight of antioxidant in a remainder of water or the mixture of water and a hydrophilic solvent and a preparation method thereof. The composition is not easily decomposed in water phase as well as in external envionment consisting of temperature change, light effect etc. because the composition is stabilized by reacting with a cationic polymer or an anionic polymer.

### TECHNICAL FIELD

The present invention relates to a composition for stabilizing epigallocatechin gallate (EGCG) in water phase and a preparation method thereof, and more particularly, to a composition containing EGCG having an improved water-in-stability wherein a polymer and an antioxidant having interaction with the EGCG are contained for the purpose of stabilizing EGCG in water phase as well as in external environment such as temperature change, light effect etc., thereby EGCG is not easily decomposed, and a preparation method thereof.

Epigallocatechin Gallate (thereinafter, this compound will be referred to “EGCG”, which is an abbreviated name) has been shown to enhance immune function of the human body, to have a strong anti-oxidation activity, and to have an excellent anti-cancer activity and a strong anti-oxidation activity in oral administration. In addition, in skin patch, EGCG promotes generation of collagen constituting cartilage, capillary, muscles etc., and prevents from skin injury by UV. Also, EGCG has previously been reported to have an effect in preventing the formation of skin wrinkles, to be very effective in improving a skin state, and to have an effective whitening effect. From these reports, EGCG has been expected to take excellent effect when appling to cosmetic compositions as well as pharmaceutical compositions, household goods, etc.

### BACKGROUND ART

However, EGCG has polyphenol chemical structure therein and has a strong anti-oxidation activity. Thus, itself is oxidized and easily decomposed by reacting sensitively with external environment such as air, oxygen, heat, light etc. The oxidation reaction of EGCG may generally be carried out by reaction with an oxidant. As a result of the reaction, a phenol group of EGCG decomposes and converts into ketone group, thus a phenyl ring of the EGCG is cutted. EGCG can be dissolved in an amount of about 4% in water phase. However, it has been reported that only a minute amount of EGCG can be used as an active ingredient in applying to pharmaceutical compositions, food compositions, cosmetic compositions etc., because EGCG is not stabilized enoughly by rapid oxidation reaction.

In order to resolve the above problem and to improve the stability, Korean Patent Publication No. 2003-75492 discloses a method for using EGCG derivatives in a cosmetic composition. Also, method for stabilizing EGCG by forming lipophilic microcapsule has been proposed. However, EGCG stabilized by the method has a poor efficiency and freshness in comparison with EGCG solution dissolved in water phase.

### DISCLOSURE OF INVENTION

Technical Problem

Thus, the present inventors have conducted extensive studies in order to resolve the above problems or drawbacks. Thereby, they found that EGCG is encapsulated when combining with polymer chain physicochemically, and EGCG is not easily decomposed although it reacts with external environment such as water, oxygen, heat, air and light when adding further a little antioxidant thereinto.

An object of this invention is to provide a composition for stabilizing effectively epigallocatechin gallate (EGCG) in water phase.

Another object of this invention is to provide a method for preparing said water-in-stable EGCG composition.

The above and other objects and features of the present invention will be apparent to the skilled in the art from the following detailed description.

Technical Solution

In order to accomplish the above objects, the present invention provides a composition for stabilizing Epigallocatechin gallate (EGCG) in water phase comprising 0.1˜25.0% by weight of Epigallocatechin gallate, 0.1˜5.0% by weight of a cationic polymer, an anionic polymer or a mixture thereof, 0.1˜10.0% by weight of an antioxidant, and water or the mixture of water and a hydrophilic solvent in a remainder.

Further, the present invention provides a method for preparing said water-in-stable EGCG composition comprising following steps of: (1) forming an aqueous Epigallocatechin gallate solution by means of dissolving Epigallocatechin gallate in water or the mixture of water and a hydrophilic solvent; (2) forming a mixture by means of adding and mixing a cationic polymer, an anionic polymer or a mixture thereof to said aqueous Epigallocatechin gallate solution at a room temperature; and (3) adding an antioxidant to the mixture, wherein the composition contains said Epigallocatechin gallate in an amount of 0.1˜25.0% by weight, said cationic polymer, said anionic polymer or said mixture thereof in an amount of 0.1˜5.0% by weight, said antioxidant in an amount of 0.1˜10.0% by weight, and water or the mixture of water and a hydrophilic solvent in a remainder.

### BEST MODE FOR CARRYING OUT THE INVENTION

A composition containing EGCG according to the present invention will be described in more detail.

A composition containing EGCG having an improved water-in-stability provided by the present invention contains EGCG, polymer(s) having interaction with the EGCG, an antioxidant, and water or the mixture of water and a hydrophilic solvent.

EGCG is dissolved in water or the mixture of water and a hydrophilic solvent to be anionic, thus reacting with a cationic polymer to formulate an stable acid-base complex. Further, cationic hydrogen of phenol group not dissociating from water or hydrophilic solvent reacts with an anionic polymer to be encapsulated. Thereby, EGCG may be stabilized in water phase. The EGCG is present in the range of preferably 0.1 to 25.0% by weight based on the total weight of the composition. When EGCG is less than 0.1% by weight, the unreacting cationic polymer interacts with the other components in cosmetics or medical supplies, so as to be educed. When EGCG is larger than 25.0% by weight, EGCG is not capable of formulating a complex by connecting with cationic polymer, so that the over-saturated EGCG is remained.

Therefore, as polymer(s) having interaction with the EGCG, a cationic polymer, an anionic polymer or the mixture thereof is used. The cationic polymer is not particularly limited as far as it interacts with the polyphenol group formed by dissolution of EGCG in water or a hydrophilic solvent. The polymer is more preferably a compound having amine group therein or a compound having a partial cation therein, which can stabilize the anion of the polyphenol group, and is harmless in human body. Examples of the cationic polymers include, but not limited thereto, chitosan, lysine, arginine, cystine, polyethylenimine, polyvinylpyrrolidone cationic copolymer, polymethylmethacrylate copolymer having quaternary ammonium, styrene copolymer having quaternary ammonium, etc. The anionic polymer is not particularly limited, as far as it interacts with a cationic hydrogen of phenol group which is not dissociated in water or a hydrophilic solvent. Examples of the anionic polymers include, but not limited thereto, polyethyleneoxide, polyethylenglycol, polypropylenglycol, polypropyleneoxide, monosaccharide, polysaccharide, cellulose, gelatin, hyaluronic acid, alginic acid, sodium alginate, starch, strach oxide and carboxymethylcellulose. The cationic polymer, the anionic polymer or the mixture thereof is present in the range of preferably 0.1 to 5.0% by weight based on the total weight of the composition. The anionic polymer is more preferably present in the same amount of the cationic polymer in order to stabilize an encapsulated membrane. When these polymers are less than 0.1% by weight, the complex is not formulated by reacting with EGCG and polymer(s). When these polymers are larger than 5.0% by weight, a part of polymers is educed so that an additional process for isolating the eductions is needed.

An antioxidant is provided for stabilizing a portion of EGCG which is not stable and remains after the additional step of polymers. Examples of the antioxidant may include, but not limited thereto, tyrosine, triptopan, Alpa-lipoic acid, vitamin C and its derivatives, vitamin E and its derivatives, vitamin A and its derivatives, sodium sulfite, sodium disulfite, etc. The antioxidant is contained in the range of preferably 0.1 to 10.0%, and more preferably 0.1 to 3.0% by weight based on the total weight of the composition. When the antioxidant is less than 0.1% by weight, the effect for stabilization of EGCG is not sufficient. When EGCG is larger than 3.0% by weight, the partial antioxidant reacts with the cationic polymer so as to formulate a complex. Thereby, EGCG is not capable of formulating a complex by reaction with the cationic polymer, thus stabilization effect by the reaction between the cationic polymer and EGCG is not sufficient.

Water or the mixture of water and a hydrophilic solvent as a solvent for dissolving EGCG is contained in remaining amount except the EGCG, the polymers and the antioxidant. The hydrophilic solvent is not particularly limited as far as it is present in polyhydric alcohol. Examples of the solvent may include, but not limited thereto, ethylene glycol, propylene glycol, diethylene glycol, dipropylene glycol, dibutylene glycol, glycerin, 1,3-butanediol, sorbitol etc. Compositions containing EGCG is easily decomposed in water only rather than in the mixture of water and a hydrophilic solvent. The hydrophilic solvent is contained, but not limited thereto, in the range of preferably 10 to 30% by weight based on the total weight of the composition.

The composition containing EGCG according to the present invention may be solidified by spray drying process or lyophilizing process. When preparing a solidified composition, an additives such as lactic acid and lactose may be added in order to dry the composition more easily. The additives is present, but not limited thereto, in the range of preferably 5 to 40% by weight based on the total weight of the composition.

The method for preparation of said water-in-stable EGCG composition according to the present invention will be described in more detail.

The method according to the present invention comprises following steps: (1) forming an aqueous Epigallocatechin gallate solution by means of dissolving Epigallocatechin gallate in water or the mixture of water and a hydrophilic solvent; (2) forming a mixture by means of adding and mixing a cationic polymer, an anionic polymer or a mixture thereof to said aqueous Epigallocatechin gallate solution at a room temperature; and (3) adding an antioxidant to the mixture.

In the step (1), EGCG is preferable to be dissolved in a hydrophilic solvent and then to be dissolved in water in order to minimize decomposition of EGCG in composition. While, when EGCG is dissolved in water only, EGCG may be decomposed by water before EGCG is stabilized.

The composition containing EGCG according to the present invention is prepared based on the idea that EGCG is dissolved in water to be anionic. EGCG reacts with a cationic polymer such as chitosan and amino acids to formulate a stabilized acid-base complex, or cationic hydrogen of phenol group not dissociating from water or hydrophilic solvent interacts with an anionic polymer such as polyethyleneoxide and polyethylenglycol to stabilize EGCG primarily. Then, an antioxidant is added into the aqueous EGCG solution to stabilize remaining unstable EGCG secondarily. Thereby, EGCG is not easily decomposed in water phase as well as in external environment such as temperature change, light effect etc.

### MODE FOR THE INVENTION

The present invention will be described in more detail by way of the following examples. However, these examples are provided for only illustration purpose and should not be construed as limiting the scope of the invention, which is properly delineated in the accompanying claims.

### EXAMPLES 1˜9 AND COMPARATIVE EXAMPLES 1˜2

TABLE 1

| | | Examples | | | | |
|---|---|---|---|---|---|---|
| Materials | | 1 | 2 | 3 | 4 | 5 |
| Hydrophilic | Glycerine | 10 | — | — | — | — |
| solvents | Diethylene glycol | — | 30 | 10 | 10 | 25 |
| | Dipropylene glycol | — | — | 20 | — | — |
| | Dibutylene glycol | — | — | — | 20 | — |
| | Sorbitol | — | — | — | — | 5 |
| Ionic | Sugar | 1 | 3 | — | — | — |
| polymers | Starch | — | — | 1 | 3 | — |
| | Polyethylene Oxide | — | — | — | — | 1 |
| | Chitosan | 1 | — | — | — | — |
| | Gelatin | — | — | — | — | 0.5 |
| | Hyaluronic Acid | — | — | 1 | — | — |
| Antioxidants | Tyrosine | 2 | — | — | — | — |
| | Alpa-lipoic acid | — | 0.5 | — | — | — |
| | Vitamin A | — | — | 1 | — | — |
| | Vitamin C | — | — | — | 2 | — |
| | Vitamin E | — | — | — | — | 2 |
| | Sodium Bisulfite | — | — | — | — | — |

| | | | | | |
|---|---|---|---|---|---|
| EGCG | 5 | 10 | 15 | 5 | 10 |
| Water | to | to | to | to | to |
| | 100 | 100 | 100 | 100 | 100 |

TABLE 2

| | | | | | | Comparative | |
|---|---|---|---|---|---|---|---|
| | | Examples | | | | Examples | |
| Materials | | 6 | 7 | 8 | 9 | 1 | 2 |
| Hydrophilic | Glycerine | 30 | 30 | 30 | 30 | 30 | 30 |
| solvents | Diethylene | — | — | — | — | — | — |
| | glycol | | | | | | |
| | Dipropylene | — | — | — | — | — | — |
| | glycol | | | | | | |
| | Dibutylene | — | — | — | — | — | — |
| | glycol | | | | | | |
| | Sorbitol | — | — | — | — | — | — |
| Ionic polymers | Sugar | — | — | — | — | 3 | — |
| | Starch | — | — | — | — | — | — |
| | Polyethylene | 3 | — | — | — | — | — |
| | Oxide | | | | | | |
| | Chitosan | — | 0.2 | — | — | — | — |
| | Gelatin | — | — | 0.2 | — | — | — |
| | Hyaluronic | — | — | — | 0.2 | — | — |
| | Acid | | | | | | |
| Antioxidants | Tyrosine | — | — | — | — | — | — |
| | Alpa-lipoic | — | — | — | — | — | — |
| | acid | | | | | | |
| | Vitamin A | — | — | — | — | — | — |
| | Vitamin C | — | 1 | 1 | 1 | — | — |
| | Vitamin E | — | — | — | — | — | — |
| | Sodium | 1 | — | — | — | — | — |
| | Bisulfite | | | | | | |

| | | | | | | |
|---|---|---|---|---|---|---|
| EGCG | 15 | 5 | 10 | 15 | 5 | 5 |
| Water | to | to | to | to | to | to |
| | 100 | 100 | 100 | 100 | 100 | 100 |

[Preparation]

Step (1) Water (or the mixture of water and a hydrophilic solvent) was bottled in a beaker or a flask, and then EGCG was dissolved in amount of the Tables 1 and 2 in water at the room temperature.

Step (2) Ionic polymers in amount of the Tables 1 and 2 were added into the solution of step (1) at the room temperature to dissolve them.

Step (3) Antioxidants in amount of the Tables 1 and 2 were added into the soultion of step (2) at the room temperature to dissolve them.

Experimental Example 1: Factor of EGCG

In compounds of the Examples 1˜9 and Comparative Examples 1˜2, primary factor of EGCG is 100. After 1 month, factor of EGCG was measured at the room temperature, 37° C. and 45° C., respectively. The results are shown in Table 3. The factor was measured with HPLC (HP 1090 manufactured by Hewlett-Packard Development Company), 280 nm Diode Array Detector and Agilent XDB C-18 column. Before the measurement of the factor, samples of the examples and comparative examples were diluted to 100 times by acetonitrile.

TABLE 3

| | | | | | | | | | | Comparative | |
|---|---|---|---|---|---|---|---|---|---|---|---|
| | Examples | | | | | | | | | Examples | |
| | 1 | 2 | 3 | 4 | 5 | 6 | 7 | 8 | 9 | 1 | 2 |
| Room | 90 | 94 | 92 | 97 | 99 | 98 | 90 | 87 | 85 | 75 | 40 |
| Temperature | | | | | | | | | | | |
| 37° C. | 85 | 90 | 92 | 96 | 98 | 97 | 87 | 84 | 81 | 65 | 31 |
| 45° C. | 79 | 88 | 87 | 92 | 94 | 91 | 83 | 79 | 78 | 57 | 17 |

As shown in the above Table 3, factors of comparative examples 1 and 2 are very low at a high temperature as well as at the room temperature. Namely, comparative examples 1 and 2 are not capable of stabilizing EGCG at the room temperature. While, examples 1˜9 of the present invention have very high factor value at the whole temperature in comparing with comparative examples 1 and 2.

### EXAMPLES 10˜18 AND COMPARATIVE EXAMPLES 3˜4

The liquid compounds produced in examples 1˜9 and comparative examples 1˜2 of said tables 1 and 2 were dried by spray drying process at the temperature range of 60˜80° C. to produce powdered EGCG compounds of examples 10˜18 and comparative examples 3˜4. In drying the compounds, lactose was contained in amount of 20% by weight based on the weight of the EGCG.

Experimental Example 2: Factor of EGCG

In compounds of the Examples 10˜18 and Comparative Examples 3˜4, primary factor of EGCG is 100. After 1 month, factor of EGCG was measured at the room temperature, 37° C. and 45° C., respectively. The results are shown in Table 4. The factor was measured with HPLC (HP 1090 manufactured by Hewlett-Packard Development Company), 280 nm Diode Array Detector and Agilent XDB C-18 column. Before the measurement of the factor, samples of the examples and comparative examples were diluted to 100 times by acetonitrile.

TABLE 4

| | | | | | | | | | | Comparative | |
|---|---|---|---|---|---|---|---|---|---|---|---|
| | Examples | | | | | | | | | Examples | |
| | 10 | 11 | 12 | 13 | 14 | 15 | 16 | 17 | 18 | 3 | 4 |
| Room | 99 | 99 | 98 | 99 | 99 | 99 | 98 | 97 | 97 | 91 | 92 |
| Temperature | | | | | | | | | | | |
| 37° C. | 98 | 97 | 97 | 97 | 98 | 98 | 97 | 96 | 95 | 88 | 89 |
| 45° C. | 95 | 96 | 93 | 95 | 94 | 97 | 96 | 95 | 91 | 85 | 86 |

As shown in the above Table 4, factor of comparative examples 3 and 4 are very improved in comparing with comparative examples 1˜2, but less than 90 at a high temperature. While, factors of examples 10˜18 of the present invention are larger than 90 at the whole temperature. Through the above experiments, it is proved that the compositons of the present invention are safe materials against an external environment.

The water-in-stable EGCG compound may be contained 1.0×10⁻⁴˜1.0×10¹% by weight based on the total weight of the composition in a cosmetic composition. Also, the EGCG compositions may be contained in medical supplies such as gauze dressing and mask pack in the same amount of the cosmetic compositon.

### INDUSTRIAL APPLICABILITY

From the results above, it is sure that the composition for stabilizing epigallocatechin gallate (EGCG) in water phase prepared in the present invention is not easily decomposed in water phase as well as in external envionment such as temperature change, light effect etc., because the composition is stabilized by reacting with a cationic polymer or an anionic polymer primarily and by reacting with an antioxidant secondarily. Also, the composition can be usefully used in a cosmetic composition, pharmaceutical compositions, household goods, etc., and has an excellent freshness because it is present as water phase.

1. A composition for stabilizing Epigallocatechin gallate (EGCG) in water phase comprising 0.1˜25.0% by weight of Epigallocatechin gallate, 0.1˜5.0% by weight of a cationic polymer, an anionic polymer or a mixture thereof, 0.1˜10.0% by weight of antioxidant, and water or the mixture of water and a hydrophilic solvent in a remainder.

2. The composition according to claim 1, which contains said hydrophilic solvent in an amount of 10˜30% by weight.

3. The composition according to claim 1 or 2, wherein said composition is solidified by spray drying process or lyophilizing process.

4. The composition according to claim 1 or 2, wherein said cationic polymer is selected from the group consisting of chitosan, lysine, arginine, cystine, polyethylenimine, polyvinylpyrrolidone cationic copolymer, polymethyl-methacrylate copolymer having quaternary ammonium and styrene copolymer having quaternary ammonium, and said anionic polymer is selected from the group consisting of polyethyleneoxide, polyethylenglycol, polypropylenglycol, polypropyleneoxide, monosaccharide, polysaccharide, cellulose, gelatin, hyaluronic acid, alginic acid, sodium alginate, starch, strach oxide and carboxymethylcellulose.

5. The composition according to claim 1 or 2, wherein said antioxidant is selected from the group consisting of tyrosine, triptopan, Alpa-lipoic acid, vitamin C and its derivatives, vitamin E and its derivatives, vitamin A and its derivatives, sodium sulfite, and sodium disulfite.

6. The composition according to claim 1 or 2, wherein said hydrophilic solvent is a polyhydric alcohol.

7. The composition according to claim 6, wherein said polyhydric alcohol is selected from the group consisiting of ethylene glycol, propylene glycol, diethylene glycol, dipropylene glycol, dibutylene glycol, glycerin, 1,3-butanediol and sorbitol.

8. A method for preparing the water-in-stable composition according to claim 1 comprising following steps of:
(1) forming an aqueous Epigallocatechin gallate solution by means of dissolving Epigallocatechin gallate in water or the mixture of water and a hydrophilic solvent;
(2) forming a mixture by means of adding and mixing a cationic polymer, an anionic polymer or a mixture thereof to said aqueous Epigallocatechin gallate solution at a room temperature; and
(3) adding an antioxidant to the mixture,
wherein the composition contains said Epigallocatechin gallate in an amount of 0.1˜25.0% by weight, said cationic polymer, said anionic polymer or said mixture thereof in an amount of 0.1˜5.0% by weight, said antioxidant in an amount of 0.1˜10.0% by weight, and water or the mixture of water and a hydrophilic solvent in a remainder.

9. The method according to claim 8, wherein said step (1) includes steps of firstly dissolving said Epigallocatechin gallate in a hydrophilic solvent and secondly dissolving said Epigallocatechin gallate in water, wherein said hydrophilic solvent is contained in an amount of 10˜30% by weight.

10. The method according to claim 8 or 9, wherein said cationic polymer is selected from the group consisting of chitosan, lysine, arginine, cystine, polyethylenimine, polyvinylpyrrolidone cationic copolymer, polymethyl-methacrylate copolymer having quaterrary ammonium and styrene copolymer having quaternary ammonium, and said anionic polymer is selected from the group consisting of polyethyleneoxide, polyethylenglycol, polypropylenglycol, polypropyleneoxide, monosaccharide, polysaccharide, cellulose, gelatin, hyaluronic acid, alginic acid, sodium alginate, starch, strach oxide and carboxymethylcellulose, and said antioxidant is selected from the group consisting of tyrosine, triptopan, Alpa-lipoic acid, vitamin C and its derivatives, vitamin E and its derivatives, vitamin A and its derivatives, sodium sulfite, and sodium disulfite, and said hydrophilic solvent is polyhydric alcohol.

11. The method according to claim 10, wherein said polyhydric alcohol is selected from the group consisiting of ethylene glycol, propylene glycol, diethylene glycol, dipropylene glycol, dibutylene glycol, glycerin, 1,3-butanediol and sorbitol.

12. A cosmetic composition containing the water-in-stable composition according to claim 1 as an active ingredient.

13. A pharmaceutical composition containing the water-in-stable composition according to claim 1 as an active ingredient.

14. A food composition containing the water-in-stable composition according to claim 1 as an active ingredient.
